(19) United States
(12) Patent Application Publication
(10) Pub. No.: US 2009/0234451 A1
(43) Pub. Date: Sep. 17, 2009

(54) METHOD AND SYSTEM FOR GRAFT LIGAMENT ATTACHMENT

(76) Inventor: Easton L. Manderson, Ashton, MD (US)

Correspondence Address: NIGEL L. SCOTT, ESQ.;SCOTT & YALLERY-ARTHUR, 7306 GEORGIA AVE, NW, WASHINGTON, DC, 20012, US

(21) Appl. No.: 12/047,252

(22) Filed: Mar. 12, 2008

(51) Int. Cl. A61F 2/08; A61B 17/56

(52) U.S. Cl. 623/13.14; 606/301

(57) ABSTRACT

Various embodiments described herein provide methods and apparatus for achieving the secure and stable fixation of a graft ligament within a bone tunnel using a screw that includes a cylindrical wall having a leading end and a trailing end, threads arranged on an outside surface of the cylindrical wall, and a plurality of portals arranged in the cylindrical wall and adapted to receive a plurality of prongs of a screwdriver.

### FIELD OF THE INVENTION

Generally, embodiments disclosed herein relate to systems and methods for attaching a graft ligament to a bone.

### BACKGROUND OF THE INVENTION

Ligaments are strong fibrous bands of tissue that serve to connect the articular extremities of bones to each other. Ligaments are typically composed of coarse bundles of dense white fibrous tissue that are disposed in a parallel or closely interlaced manner. The fibrous tissue of ligaments is pliant and flexible, but not significantly extensible (stretchable). Ligaments may be torn or ruptured as a result of accidents. The ligaments of the knee joint are especially susceptible to injury and may be repaired using arthroscopic surgical procedures.

For example, in the human knee, the anterior cruciate ligament (ACL) and posterior cruciate ligament (PCL) extend between the top end (proximal end) of the tibia and the bottom end (distal end) of the femur. The ACL and PCL cooperate, together with other ligaments and soft tissue, to provide both static and dynamic stability to the knee. The rupture or tearing of the ACL is a common occurrence among participants of sports activities such as football and skiing.

A rupture of the ACL generally does not heal spontaneously. Loss of the function of this stabilizing ligament may cause mechanical damage to other intra-articular tissues and may impair daily living activities, such as walking and bending the knee. Various surgical procedures have been developed for reconstructing the ACL to restore normal function to the knee. ACL reconstruction may be done by intra-articular or extra-articular methods. The intra-articular method seeks to duplicate the anatomic position and function of the ACL. The extra-articular method seeks primarily to reduce harmful force normally restrained by an uninjured ACL. The ideal reconstruction should be minimally invasive and allow early mobilization to preserve knee motion and avoid stiffness without failure of the construct. Furthermore, the extra-articular cortex and adjacent soft tissues should not be disturbed to reduce the chance of stress-riser fractures, quadriceps weakness, and heterotopic bone-formation.

In many instances, the ACL may be reconstructed using an intra-articular method by replacing the ruptured ACL with a graft ligament. The graft ligament may be a ligament or tendon which is harvested from elsewhere in the patient, such as a hamstring muscle (or any of the Semitendinosus, Semimembranosus, and Biceps femoris muscles), or may be a synthetic device. To connect the graft ligament to the bones at issue, bone tunnels are typically formed in the top end of the tibia and the bottom end of the femur, with one end of the graft ligament being positioned in the femoral tunnel and the other end of the graft ligament being positioned in the tibial tunnel. The two ends of the graft ligament are anchored in place in various ways known in the art so that the graft ligament extends between the femur and the tibia in substantially the same way, and with substantially the same function, as the original ACL to restore normal function to the knee.

Various methods of anchoring a graft ligament to the bone are known, such as staples, suture over buttons, and interference screw fixation. Staples and suture buttons are disadvantageous because they often do not provide a sufficiently strong attachment to withstand the normal tensile loads to which they are normally subjected. For example, with suture button fixation, the strand of suture coupling the button and the graft ligament becomes the weakest link in the chain, and if the suture breaks, the graft ligament will detach.

Another method of anchoring a graft ligament to a bone includes inserting the graft ligament into the opening in the femur and looping the graft ligament over a post inserted transversely into the bone. However, this method requires two holes to be drilled into the bone and requires that a deep intermuscular incision be made through soft tissue down to the bone to insert the transverse post, which requires additional recovery time. Furthermore, wrapping a graft ligament around a conventional post may cause the graft ligament to slide back and forth on the post during movement of the joint in what is known as the “windshield wiper effect.” The windshield wiper effect may abrade the graft ligament due to friction against the post and may cause failure of the graft ligament.

Another method of anchoring a graft ligament to a bone uses an interference screw to pin the graft ligament against the side of the femoral tunnel. However, interference screws are known to abrade the graft ligament with the screw threads and risk failure of the graft ligament fixation by rupturing. The probability of failure is exacerbated by the use of a metal screw with graft ligaments. Furthermore, the use of a biodegradable screw in place of a metal screw does not sufficiently reduce the risk of failure because the very nature of interference fixation requires high friction and tight apposition of the hard screw threads against the graft ligament, which can themselves cause abrasive damage. Furthermore, biodegradable screws have a higher rate of drive failure under a torque force sufficient to produce effective interference fixation.

What is needed is a method and system to attach a graft ligament to bone without the drawbacks of the prior art.

### BRIEF SUMMARY OF THE INVENTION

The various embodiments described herein provide methods and apparatus for achieving the secure and stable fixation of a graft ligament within a bone tunnel.

One embodiment described herein provides a screw including a cylindrical wall having a leading end and a trailing end, threads arranged on an outside surface of the cylindrical wall, and a plurality of portals arranged in the cylindrical wall and adapted to receive a plurality of prongs of a screw driver. In other embodiments, the screw may include a cannula that extends from a leading edge to a trailing edge of the screw.

In another embodiment, the screw further includes a crossbar arranged at the leading end of the screw. Embodiments of the crossbar include a handle having a first end and a second end, and end portions connected to the first end and the second end of the handle, where the end portions are wider than the handle.

Another embodiment described herein provides a screwdriver adapted to drive the screws. The screwdriver includes a handle, a shaft, and a plurality of prongs adapted to fit the plurality of portals of the screws.

Another embodiment described herein provides a hood adapted to cover at least a portion of the prongs of the screwdriver. The hood includes an opening and is formed of a material that is perforated when the plurality prongs of the screwdriver are inserted into the plurality of portals of the screw.

Another embodiment described herein provides a method of attaching a graft ligament to a bone and includes forming a tunnel in the bone, providing a screw comprising, a cylindrical wall having an outer surface and an inner surface, a cannula extending from a leading end and a trailing end of the screw, threads arranged on an outside surface of the cylindrical wall, a plurality of portals arranged in the cylindrical wall and adapted to receive a plurality of prongs of a screw driver, and a crossbar arranged at the leading end of the screw, arranging a graft ligament through the cannula of the screw and around the crossbar, and screwing the screw and the graft ligament into the tunnel in the bone.

Another embodiment described herein provides a method of attaching a graft ligament to a bone and includes forming a tunnel in the bone, providing a screw comprising a cylindrical wall having an outer surface and an inner surface, threads arranged on an outside surface of the cylindrical wall, and a plurality of portals arranged in the cylindrical wall and adapted to receive a plurality of prongs of a screw driver, providing a graft ligament in the tunnel and screwing the screw into the tunnel to fix the graft ligament into the tunnel.

### BRIEF DESCRIPTION OF THE DRAWINGS

FIG. 1A is a diagram of a cannulated screw according to an embodiment described herein.

FIG. 1B is a diagram of a cannulated screw according to an embodiment described herein.

FIG. 2 is a diagram of a screwdriver according to an embodiment described herein.

FIG. 3 is a diagram of a cannulated screw according to an embodiment described herein.

FIG. 4 is a diagram of a screwdriver according to an embodiment described herein.

FIG. 5 is a diagram of the screwdriver covered by a purse stringed hood according to an embodiment described herein.

FIG. 6A is a diagram of a crossbar according to an embodiment described herein.

FIG. 6B is a diagram of a crossbar according to an embodiment described herein.

FIG. 6C is a diagram of a crossbar according to an embodiment described herein.

FIG. 7 is a diagram of a cannulated screw according to an embodiment described herein.

FIG. 8A is a diagram of a cannulated screw having a crossbar and a graft ligament threaded through the cannulated screw and around the crossbar according to an embodiment of a method describe herein.

FIG. 8B is a diagram of a cannulated screw having a crossbar and a graft ligament threaded through the cannulated screw and around the crossbar according to an embodiment of a method describe herein.

FIG. 8C is a diagram of a cannulated screw having a crossbar and a graft ligament threaded through the cannulated screw and around the crossbar according to an embodiment of a method describe herein.

FIG. 8D is a diagram of a cannulated screw having an allograft bone and a graft ligament threaded through the cannulated screw according to an embodiment of a method describe herein.

FIG. 9 is diagram of a human knee prepared for insertion of a cannulated screw according to an embodiment of a method described herein.

FIG. 10 is a diagram of a cannulated screw, a crossbar, and a graft ligament being inserted into a femur using a screwdriver according to an embodiment of a method described herein.

FIG. 11 is a diagram of a cannulated screw, a crossbar, and a graft ligament inserted into a femur according to an embodiment of a method described herein.

FIG. 12A is a diagram of an interference screw according to an embodiment described herein.

FIG. 12B is a diagram of an interference screw according to an embodiment described herein.

FIG. 13 is a diagram of an interference screw according to an embodiment described herein.

FIG. 14 is diagram of a tibia prepared for insertion of an interference screw according to an embodiment of a method described herein.

FIG. 15 is a diagram of two corticocancellous bone grafts.

FIG. 16 is a diagram of two corticocancellous bone grafts inserted into a tibia according to an embodiment of a method described herein.

FIG. 17 is a diagram of an interference screw being inserted into a tibia using a screwdriver according to an embodiment of a method described herein.

FIG. 18 is a diagram of an interference screw inserted into a tibia according to an embodiment of a method described herein.

FIG. 19 is a diagram of an interference screw and injectable or particulate graft inserted into a tibia according to an embodiment of a method described herein.

FIG. 20 is a diagram of a chisel according to an embodiment described herein that may be used to remove a screw from a bone.

### DETAILED DESCRIPTION OF THE INVENTION

In the following detailed description, reference is made to the accompanying drawings which form a part hereof, and in which are shown by way of illustration of specific embodiments in which the invention may be practiced. These embodiments are described in sufficient detail to enable those of ordinary skill in the art to make and use them, and it is to be understood that structural, logical or procedural changes may be made to the specific embodiments disclosed herein.

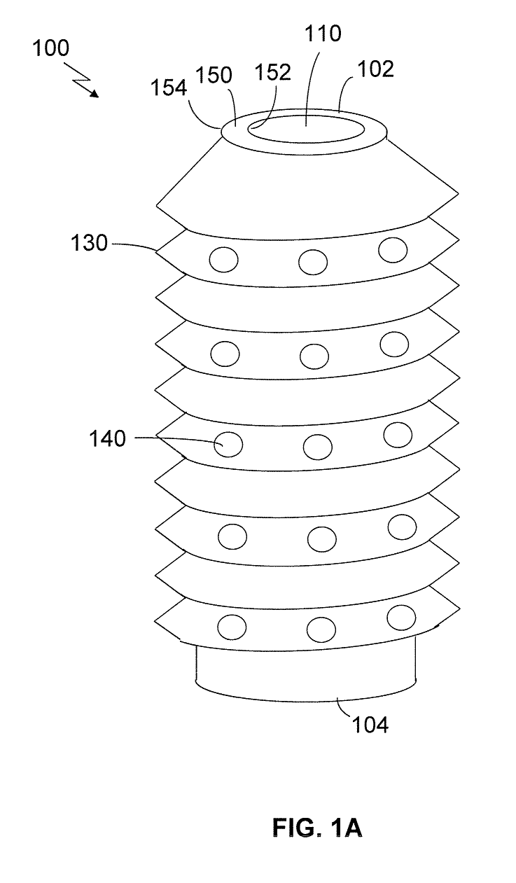

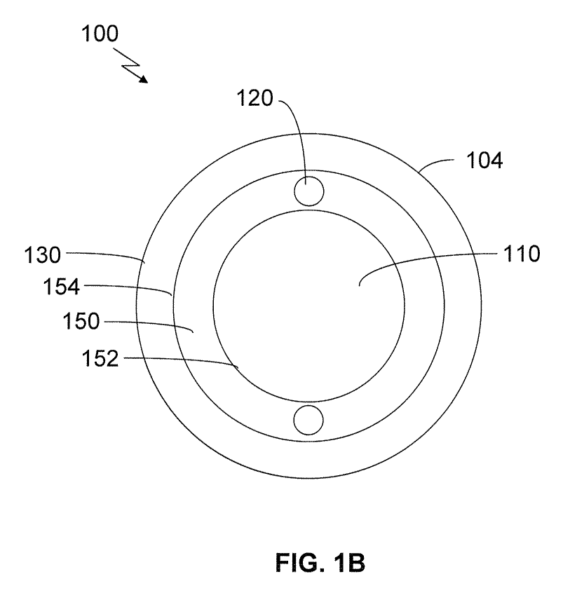

FIG. 1A is a side view and 1B is a trailing end view of a cannulated screw 100 having a cannula 110 extending from a leading end 102 to a trailing end 104 of the cannulated screw 100 through a cylindrical wall 150 having an inner surface 152 and an outer surface 154, two prong portals 120, threads 130, and optional perforations 140. The cannulated screw 100 may be formed of metal, such as stainless steel or titanium, a biodegradable material, such as a biodegradable polymer, or a combination of the two forgoing materials.

The cannulated screw 100 may be sized to a width and length to best fit a particular bone for a particular application. In one embodiment, the cannulated screw 100 may have an outer diameter between about 6 to about 15 mm, and more particularly, between about 8 to about 12 mm. In another embodiment, the cannulated screw 100 may have a length of about 15 to 30 mm, and more particularly between about 18 to 22 mm.

The threads 130 are wrapped around the cylindrical wall 150 and are sized to affix the cannulated screw 100 firmly into cancellous bone in a bone tunnel. In one embodiment, the threads 130 may be wrapped around the cylindrical wall 150 about 5 to about 15 times. The trailing end 104 of the cannulated screw 100 may be smooth, i.e., may lack threads 130, to prevent abrasion to the proximal intra-articular portion of the graft ligament that may come in contact with the trailing end 104 of the cannulated screw 100.

The leading end 102 and the trailing end 104 of the cannulated screw 100 should lack sharp edges that might cut through a graft ligament. In one embodiment, the leading end 102 and trailing end 104 may be curved so that they lack sharp edges. In another embodiment, the leading end 102 and trailing end 104 may be rounded.

The perforations 140 are optionally arranged in the cannulated screw 100 to allow osteo-integration of the soft tissue graft within the femoral tunnel. The number, size, shape, placement of the perforations 140 may be varied, but should be arranged in such a way so as to allow osteo-integration of the soft tissue graft without significantly weakening the integrity of the screw.

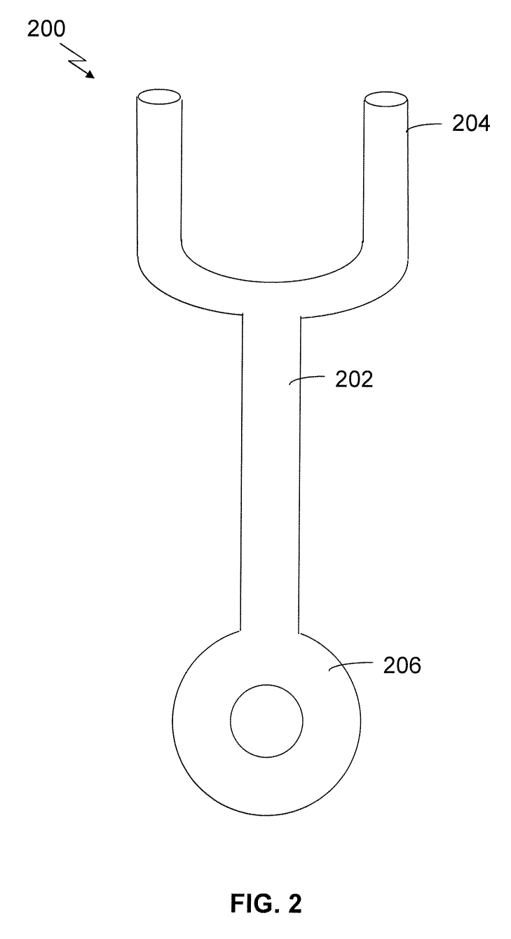

As shown in FIG. 1B, the prong portals 120 are arranged at the trailing end 104 of the cannulated screw 100 to accept prongs from a screwdriver (FIG. 2) to drive the cannulated screw 100 into a tunnel formed in a bone. The prong portals 120 extend from the trailing end 104 of the cannulated screw 100 into the cylindrical wall 150. In various embodiments, the prong portals 120 may extend to varying depths within the cylindrical wall 150. In one embodiment, the prong portals 120 do not have an exit at the leading end 102 of the cannulated screw 100. In another embodiment, the prong portals 120 do have an exit at the leading end 102 of the cannulated screw 100.

FIG. 2 is a diagram of a screwdriver 200 according to an embodiment described herein and adapted to be used with the cannulated screw 100. The screwdriver 200 includes a shaft 202, a handle 206, and two prongs 204. The handle 206 of the screwdriver 200 may be of variable length, but should be long enough to seat a cannulated screw 100 into the end of a bone tunnel. The prongs 204 are adapted to fit into the prong portals 120 of the cannulated screw 100 to allow the cannulated screw 100 to be screwed into a bone tunnel. The length of the prongs 204 may match the depth of the prong portals 120 so that the prongs 204 may be fully seated within the prong portals 120.

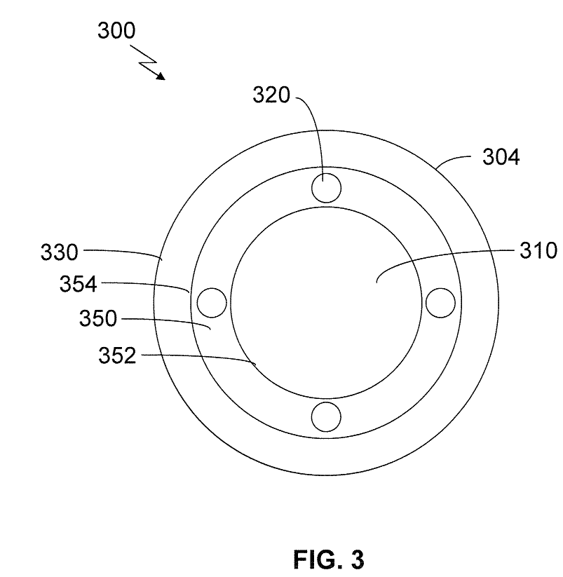

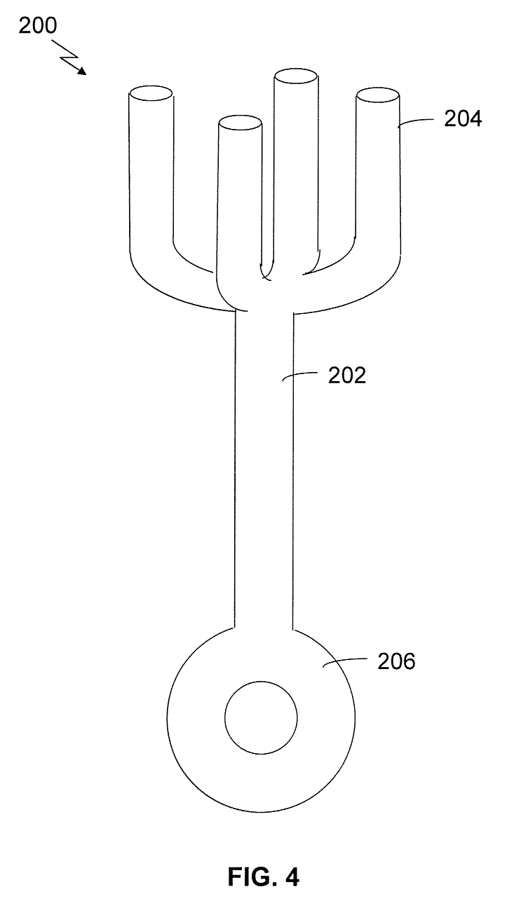

In various embodiments, the cannulated screw 100 may include more than two prong portals 120, for example, three, four, five, six, or more prong portals 120, to more evenly distribute the torque force from a screwdriver (FIG. 4) needed to drive the cannulated screw 100 over a larger drive function surface. FIG. 3 is a diagram of a trailing end of a cannulated screw 300 having a cylindrical wall 350 having an inner surface 352 and an outer surface 354, a cannula 310 extending through the cannulated screw 300 from a leading end 302 to a trailing end 304, four prong portals 320, and threads 330.

FIG. 4 is a diagram of a screwdriver 400 according to an embodiment described herein. The screwdriver 400 includes a shaft 402, a handle 406, and four prongs 404. The four prongs 404 of the screwdriver 400 are adapted to fit into the four prong portals 320 of the cannulated screw 300. By more evenly distributing the torque force from the screwdriver 400, the cannulated screw 300 will be better able to tolerate the torque force applied to drive the cannulated screw 300 to interference fixation without failure. This is especially important for a cannulated screw formed of biodegradable material, because biodegradable material is know to structurally fail more readily than metal screws of the same type.

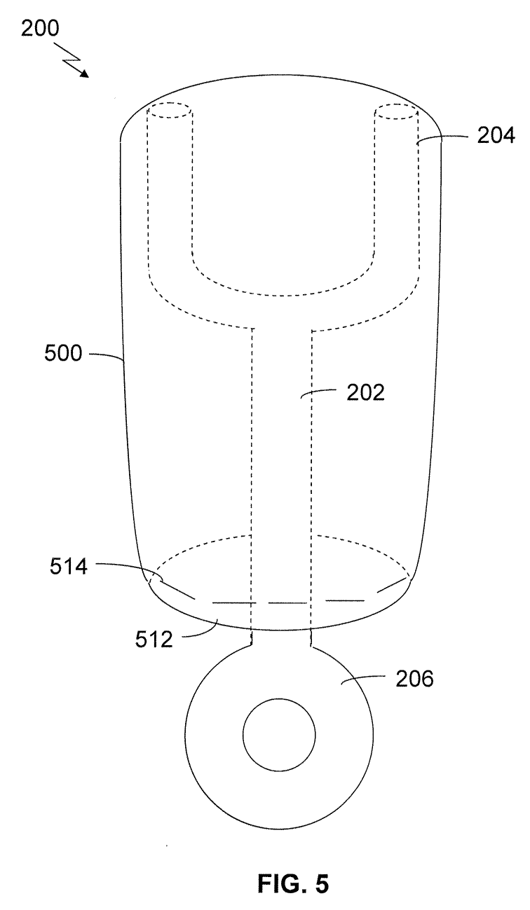

FIG. 5 is a diagram of the screwdriver 200 of FIG. 2 covered by a purse-stringed hood 500. The hood 500 includes an opening 512 and a purse-string 514 arranged around the opening 512 to cinch the hood 500 to the screwdriver 200. The hood 500 is adapted to fit over the end of the screwdriver 200 to cover the prongs 204. The hood 500 may be made of material of a type and thickness so that the hood is easily perforated by the prongs 204 of the screwdriver 200 when the prongs 204 are pushed into the pong portals 220. For example, the hood 500 may be made of synthetic polymers, such as latex, or natural polymers, such as rubber.

If the screwdriver 200 is removed from a bone tunnel before finally seating the cannulated screw 100 in the bone, the screwdriver 200 may be replaced by covering it with the hood 500 and reinserting the screwdriver 200 into the tunnel. The hood 500 will prevent the prongs 204 from being entangled with soft tissue on reinsertion. By applying gentle force, the prongs 204 will perforate the hood 500 to allow penetration of the prongs 204 into the prong portals 120. Alternately, the hooded screwdriver 200 may be reinserted into a bone tunnel, such as a femoral tunnel, from the anterior medial portal over a guidewire inserted into the cannulated screw 100 and/or femoral tunnel.

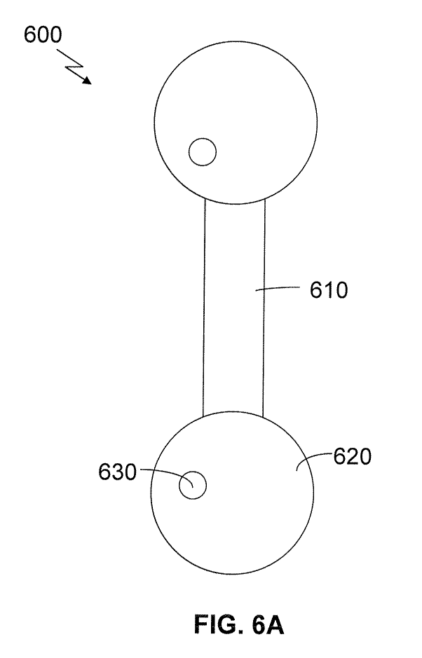

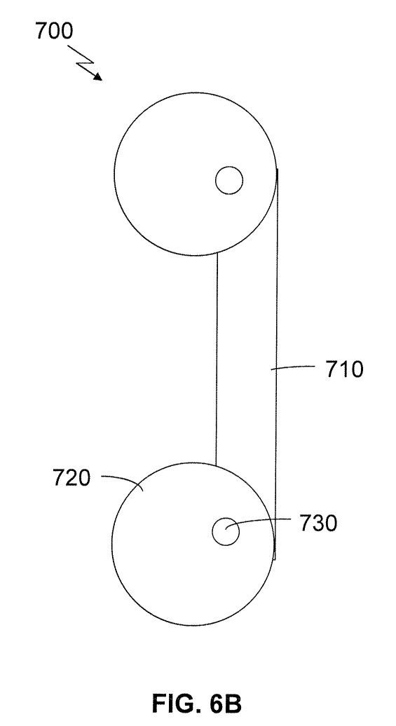

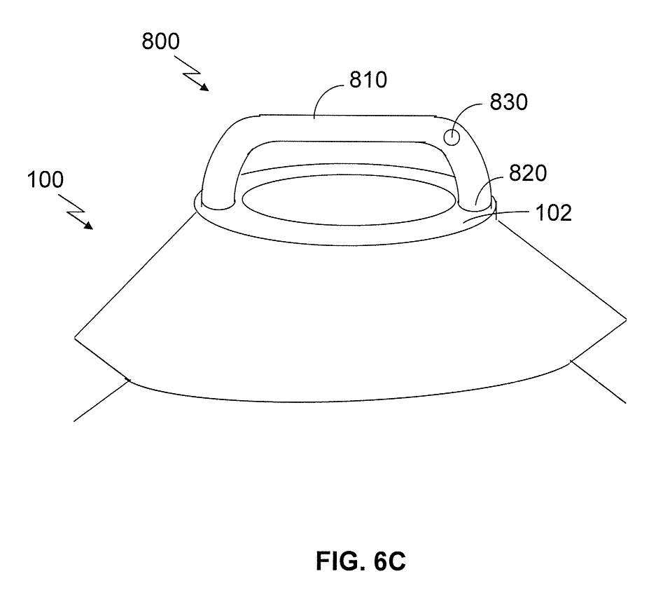

FIG. 6A is a diagram of a crossbar 600 to be used in conjunction with the cannulated screw 100 according to an embodiment. The crossbar 600 includes a handle 610, end portions 620 that are wider than the handle 610 arranged at both ends of the handle 610, and optional apertures 630 arranged in the end portions 620. The crossbar 600 may be arranged at the leading edge 102 of the cannulated screw 100 so that a graft ligament may be passed through the cannula 110 and wrapped around the handle 610 of the crossbar 600. The crossbar 600 may be formed of metal, such as stainless steel or titanium, a biodegradable material, such as a biodegradable polymer, or a combination of the two forgoing materials.

In the embodiment shown in FIG. 6A, the crossbar 600 is shaped much like a type of exercise equipment commonly known as a “dumbbell,” with the end portions 620 having a spherical shape and centered on the handle 610. In other embodiments, the end portions 620 may have other shapes, such as oval, rectangular, and the like. The handle 610 of the crossbar 600 should be long enough so that the end portions 620 fit over the outer surface 154 of the cylindrical wall 150 so that the crossbar 600 does not move laterally with respect to the cannulated screw 100. The optional apertures 630 may be used to fasten a graft ligament to the crossbar 600 using a suture. The crossbar 600 should not be longer than the outer diameter of the cannulated screw 100 and should not have sharp edges that may cut through a graft ligament.

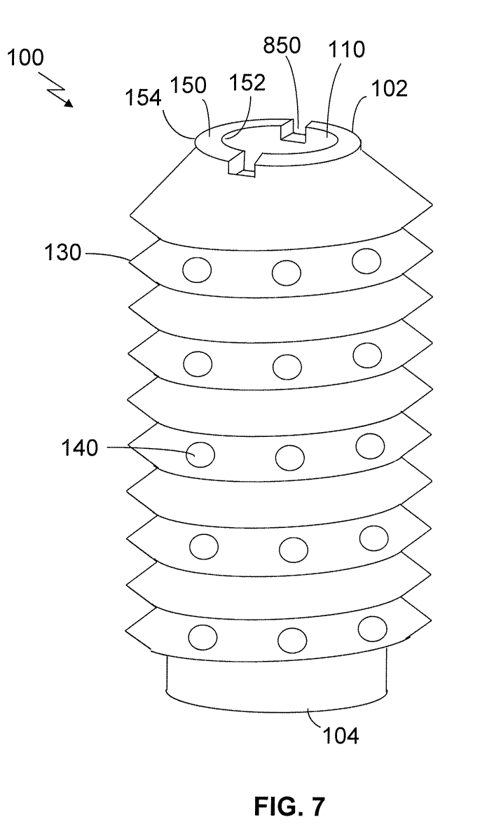

FIG. 6B is a diagram of a crossbar 700 according to another embodiment that includes a handle 710, end portions 720, and optional apertures 730 arranged in the end portions 720. In the embodiment shown in FIG. 7, the crossbar is shaped like a dumbbell, with end portions 720 having a spherical shape and offset from the center of the handle 710. The handle 710 is arranged to position the looped end of a graft ligament further from the leading end 102 of the cannulated screw 100 and closer to the end of a bone tunnel for osteo-integration.

FIG. 6C is a diagram of a crossbar 800 according to another embodiment. The crossbar 800 includes a handle 810, optional apertures 830, and end portions 820 that are fixed to the leading end 102 of the cannulated screw 100. The crossbar 800 will not slip or move with respect to the cannulated screw 100 because it is firmly affixed to the leading end 102. The shape of the crossbar 800 may be modified so long as no sharp edges are present that may cut through a graft ligament.

FIG. 7 is a side view of a cannulated screw 100 having grooves 850 arranged in the cylindrical wall 150 at the leading edge 102 that are adapted to receive a crossbar 600, 700. The grooves 850 will prevent the crossbar 600, 700 from moving in a transverse direction with respect to the cannulated screw 100.

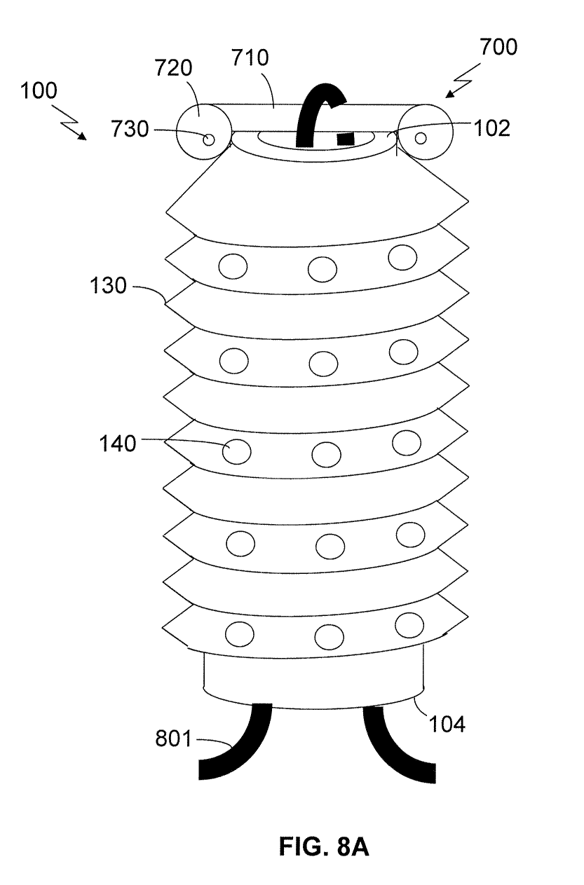

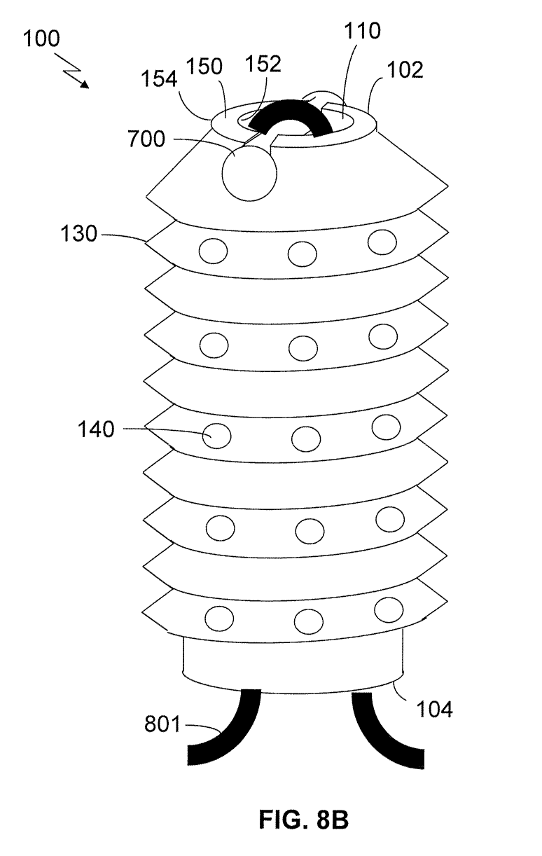

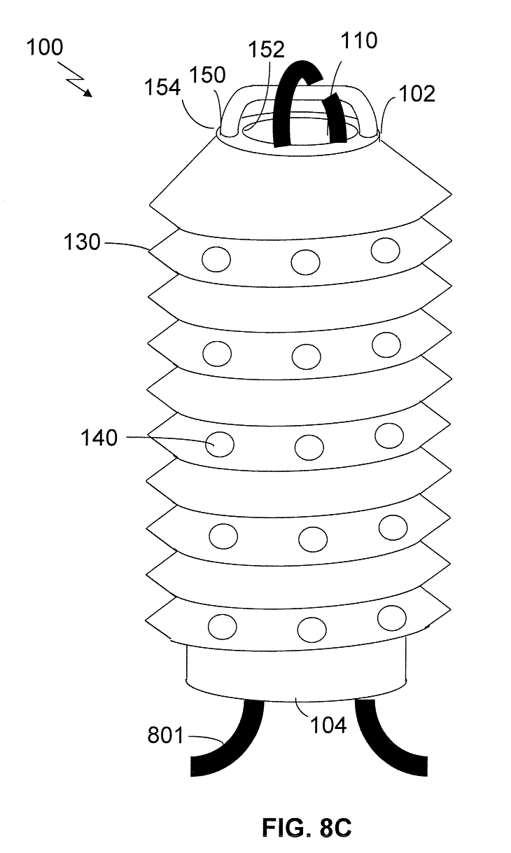

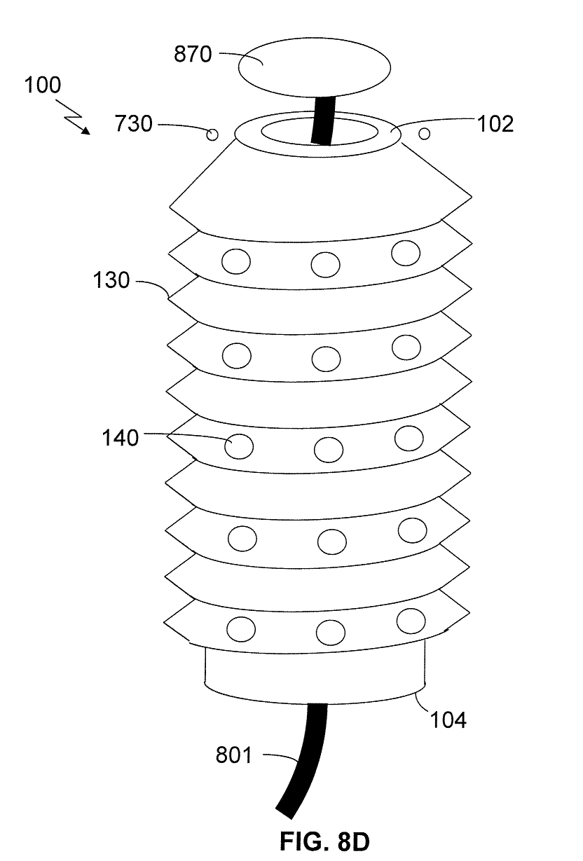

A method of installing a graft ligament within a femoral tunnel using the cannulated screws and crossbars of the various embodiments is described below. FIG. 8A is a diagram of a cannulated screw 100 having a crossbar 700 arranged transversely to the length of the cannulated screw 100. A looped end of a graft ligament 801 is threaded through the cannula 110 at the trailing end 104 of the cannulated screw 100 and out the leading end 102. The crossbar 700 is inserted through the looped graft ligament 801 at the leading end 102 of the cannulated screw 100 so that the graft ligament 801 rests on the handle 710 of the crossbar 700. The crossbar 700 entraps the graft ligament 801 and prevents it from pulling back through the cannula 110. The handle 710 of the crossbar 700 is of a length so that the end portions 720 extend over the edges of the cannulated screw 100 to keep the crossbar 700 from moving transversely to the length of the cannulated screw 100.

FIG. 8B is a diagram of a cannulated screw 100 having grooves 850 arranged in the cylindrical wall 150 at the leading edge 102 and a crossbar 700 positioned in the grooves 850. The grooves 850 prevent the crossbar 700 from moving in a transverse direction with respect to the cannulated screw 100. A looped end of a graft ligament 801 is threaded through the cannula 110 of the cannulated screw 100 and around the crossbar 700.

FIG. 8C is a diagram of a cannulated screw 100 including a crossbar 800 having end portions 820 that are fixed to the leading end 102 of the cannulated screw 100. A looped end of a graft ligament 801 is threaded through the cannula 110 of the cannulated screw 100 and around the crossbar 800.

FIG. 8D is a diagram of a cannulated screw 100 including an allograft bone 870 having a ligament 801 attached. The allograft bone 870 may take the place of a crossbar in fixing a ligament 801 to the cannulated screw 100. The allograft bone 870 is larger than the cannula 110 and prevents the graft ligament 801 from pulling back through the cannulated screw 100.

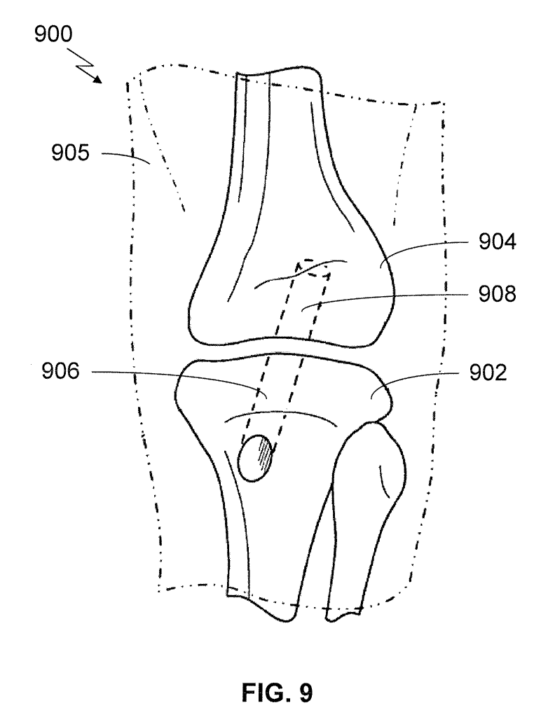

FIG. 9 is diagram of a knee 900 prepared for insertion of the cannulated screw 100, crossbar 700, and graft ligament 801. The knee 900 includes a tibia 902 and a femur 904 surrounded by soft tissue 905. Incisions are made in the soft tissue 905 surrounding the tibia 902 to expose the tibia 902. Next, a suitable tool, such as a pneumatic or electric drill or a “reamer” tool, or other such equivalent medical device, is used to drill a tibial tunnel 906 in the tibia 902. Next, a femoral tunnel 908 is drilled into the femur 904 so that the femoral tunnel 908 and the tibial tunnel 906 are aligned. The femoral tunnel 908 is a blind tunnel, which terminates below the surface of the femur 904.

The size of the tibial tunnel 906 and the femoral tunnel 908 depends upon the size of the bones and the size of the graft ligament to be implanted. The tibial tunnel 906 and femoral tunnel 908 may be drilled to any required diameter, but are generally between about 5 and 18 millimeters.

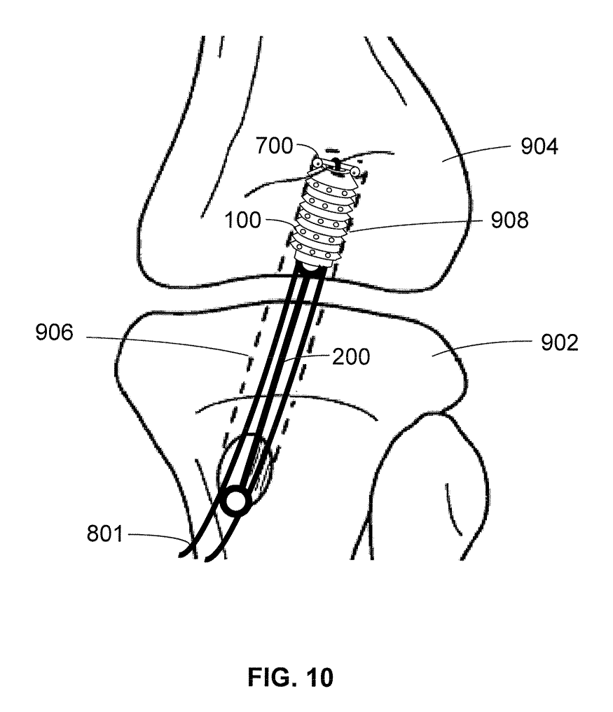

As shown in FIG. 10, the cannulated screw 100, crossbar 700, and entrapped graft ligament 801 are inserted into the knee 900 using a screwdriver 200. The ratio of the diameter of the femoral tunnel 908 to the outside diameter of the cannulated screw 100 may vary according to a doctor's preference of tightness of fit. In one embodiment, the ratio may be 1:1. In another embodiment, the ratio may be 9:10.

The prongs 204 of the screwdriver 200 are inserted into the prong portals 120 of the cannulated screw 100. The cannulated screw 100 is then inserted through the tibial tunnel 906 into the femoral tunnel 908. In one embodiment, the tibial tunnel 906 may be drilled wider than the femoral tunnel so that the cannulated screw 100 may be pushed through the tibial tunnel 906 without turning the cannulated screw 100. The cannulated screw 100 is then screwed into the femoral tunnel 908 to seat the cannulated screw 100 into the femoral tunnel 908. In another embodiment, the tibial tunnel 906 and the femoral tunnel 908 may be the same size and the cannulated screw 100 is screwed through the tibial tunnel 906 and into the femoral tunnel 908. In other embodiments, the screwdriver 200 may be inserted into the femur 904 from another suitable portal.

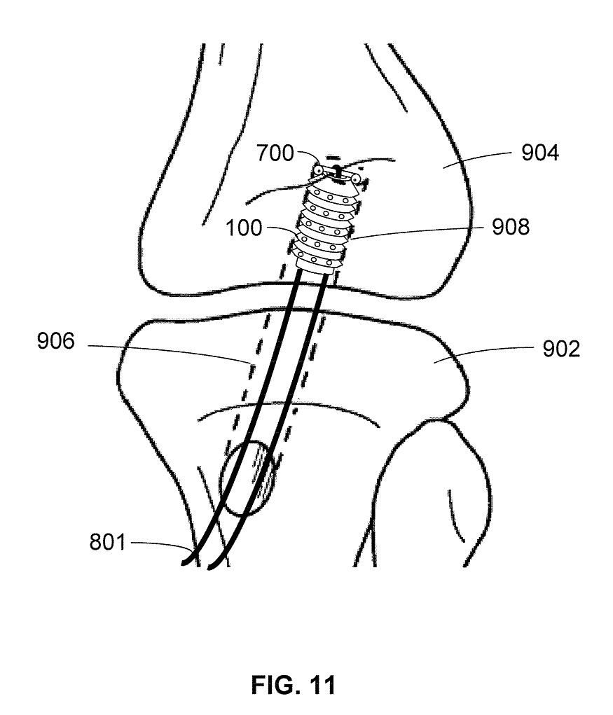

As shown in FIG. 11, the screwdriver 200 is removed and the cannulated screw 100, crossbar 700, and graft ligament 801 remain implanted in the femoral tunnel 908. In one embodiment, the cannulated screw 100 may be seated so that it fills up most of the femoral tunnel 908. In another embodiment, the cannulated screw 100 may protrude from the end of the femoral tunnel 908.

The exposed graft ligament 801 above the crossbar 700 may be pressed against the cancellous bone inside the femoral tunnel 908 to allow osteo-integration. The graft ligament 801 is thus protected within the cannulated screw 100. The cannulated screw 100 will support the interior wall of the femoral tunnel 908 to increase the stiffness of the overall construct while simultaneously allowing osteo-integration of the graft ligament 801.

The graft ligament 801 will contact the inner wall of the femoral tunnel 908 to help to maximize the tunnel fill and improve biological activity within the femoral tunnel 908. These features are designed to reduce axial and longitudinal motion, e.g., the windshield wiper effect, of the graft ligament 801 within the femoral tunnel 908, especially after cycling forces performed in the postoperative rehabilitation period, thereby reducing lysis and tunnel enlargement.

As discussed above, the trailing end 104 of the cannulated screw 100 is smooth and acts as a grommet or “bushing” to prevent abrasion to the proximal intra-articular portion of the graft ligament 801 in contact with the trailing end 102 of the cannulated screw 100. This type of fixation improves the strength of the fixation and may reduce slippage to cyclical loading in the post-operative rehabilitation period.

The embodiments described above provide an improvement in fixation of ACL graft construct, and may allow early and accelerated rehabilitation, in part due to the fact that there is no need to make a transverse hole through the femur 904 to insert a crossbar, which requires incisions to be made through the nearby external soft tissue. The embodiments described above also allow the use of a biodegradable or metal cannulated screw 100 to attach a graft ligament 801 without concern for drive failure or graft abrasion until biologic incorporation, i.e., osteo-integration of the graft ligament 801 within the femoral tunnel 908, is complete. The ability to use a metal cannulated screw 100 without the fear of graft abrasion may reduce later failure due to degraded debris and lysis within the femoral tunnel 908.

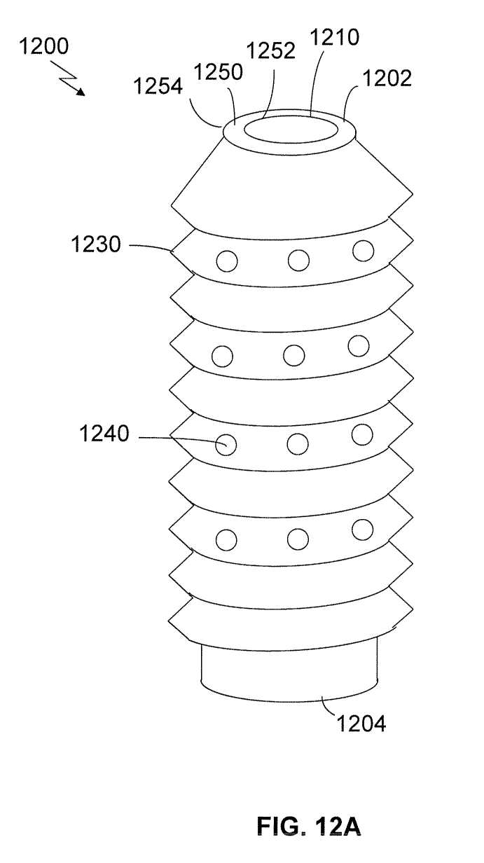

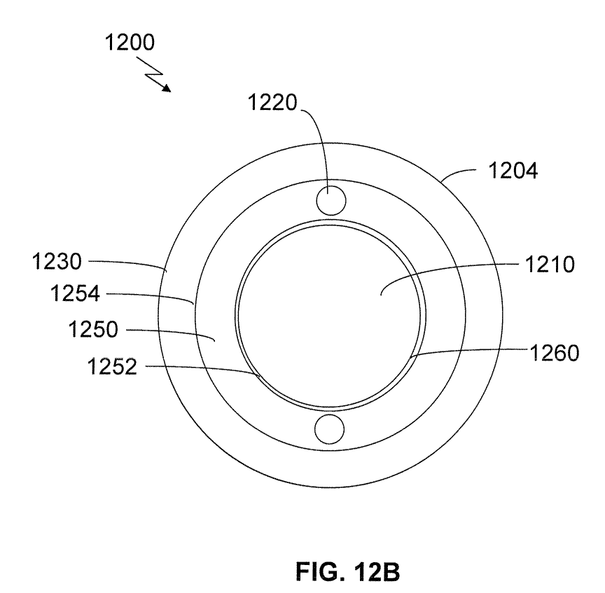

FIG. 12A is a side view and FIG. 12B is a trailing end view of an interference screw 1200 having a cylindrical wall 1250 having an outer surface 1254, an optional cannula 1210 having an inner surface 1252 extending through the interference screw 1200 from a leading end 1202 to a trailing end 1204, threads 1230 arranged on the outer surface 1254 of the cylindrical wall 1250, two prong portals 1220, optional perforations 1240, and optional threads 1260 arranged on the inner surface 1252 at the trailing end 1204 of the cylindrical wall 1250. The interference screw 1200 may be formed of metal, such as stainless steel or titanium, or may be formed of a biodegradable material, such as a biodegradable polymer, or a combination of the two foregoing materials.

The interference screw 1200 may be sized to a width and length to best fit a particular bone for a particular application. In one embodiment, the interference screw 1200 may have an outer diameter between about 4 mm to about 16 mm, and more particularly, between about 8 mm to about 12 mm. In another embodiment, the cannulated screw 100 may have a length of about 15 to 25 mm and more particularly between about 18 to 22 mm.

The threads 1230 are wrapped around the cylindrical wall 1250 and are sized to affix the interference screw 1200 firmly into cancellous bone in a bone tunnel. In one embodiment, the threads 1230 may be wrapped around the cylindrical wall 1250 about 5 to about 15 times. The outer surface 1264 of the trailing end 1204 may be smooth to prevent abrasion to the proximal intra-articular portion of the graft ligament 801 that may come in contact with the trailing end 1204 of the interference screw 1200.

The leading end 1202 and the trailing end 1204 of the interference screw 1200 should lack sharp edges that might cut through a graft ligament. In one embodiment, the leading end 1202 and trailing end 1204 may be curved so that they lack sharp edges. In another embodiment, the leading end 1202 and trailing end 1204 may be rounded.

The perforations 1240 are optionally arranged in the interference screw 1200 to allow osteo-integration of a graft ligament within a bone tunnel. The number, size, shape, placement of the perforations 1240 may be varied, but should be arranged in such a way so as to allow osteo-integration of the soft tissue graft without significantly weakening the integrity of the interference screw 1200. The interference screw 1200 may optionally embody a whole or partial cannula or lumen 1210, or may have a solid core.

As shown in FIG. 12B, the prong portals 1220 are arranged to accept prongs from a screwdriver, such as the screwdriver 200 in FIG. 2, to drive the interference screw 1200 into a bone tunnel. The prongs 204 of the screwdriver 200 are adapted to fit into the prong portals 1220 of the interference screw 1200. In various embodiments, the prong portals 1220 may extend to varying depths within the cylindrical wall 1250. In one embodiment, the prong portals 1220 do not have an exit at the leading end 1202 of the interference screw 1200. In another embodiment, the prong portals 1220 do have an exit at the leading end 1202 of the interference screw 1200.

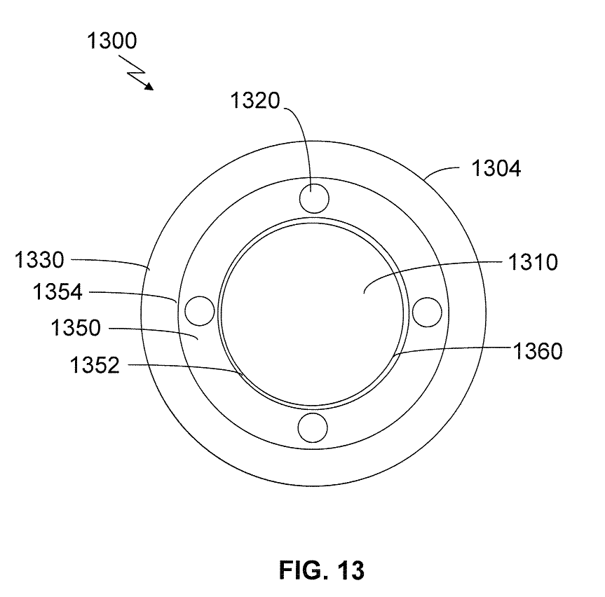

In various embodiments, the interference screw 1200 may include more than two prong portals 1220, for example, three, four, five, six, or more prong portals 1220, to more evenly distribute the torque force from a screwdriver, such as the screwdriver 400 of FIG. 4, needed to drive the interference screw 1200 over a larger drive function surface. FIG. 13 is a diagram of an interference screw 1300 having a wall 1360 having an outer surface 1364, an optional cannula 1310 having an inner surface 1362 extending through the interference screw 1300 from a leading end 1302 to a trailing end 1304, threads 1330, optional perforations 1340, and optional threads 1350 arranged on the inner surface 1362 at the trailing end 1304 of the wall 1360. The prongs 404 of the screwdriver 400 are adapted to fit into the prong portals 1320 of the interference screw 1300. By more evenly distributing the torque force from a screwdriver 400, the interference screw 1300 will be better able to tolerate the torque force applied to drive the interference screw 1300 to interference fixation without failure. In one embodiment, one screwdriver 200, 400 may be adapted to fit the prong portals 120, 320 of a cannulated screw 100, 300 and the prong portals 1220, 1320 of an interference screw 1200, 1300.

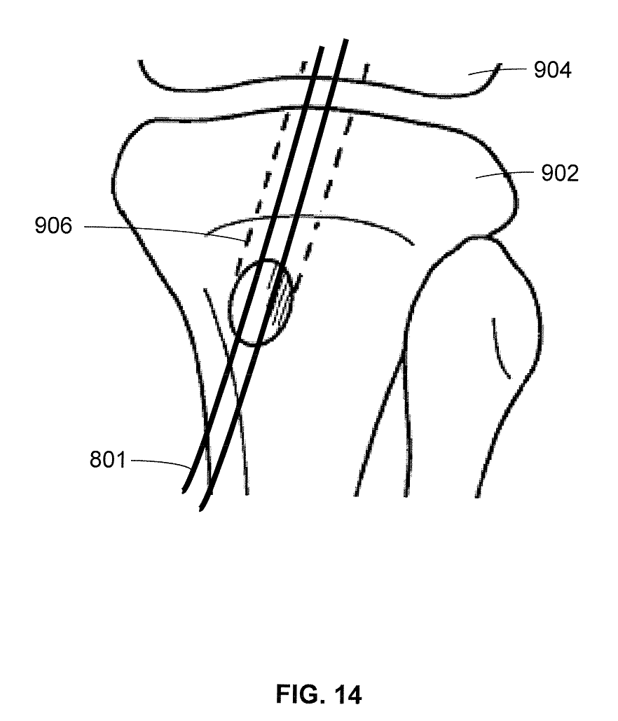

A method of installing a graft ligament within a tibial tunnel using an interference screw of the various embodiments is described below. After a graft ligament 801 has been attached to the femur 904, for example, by the method described above or by other methods, the graft ligament 801 will extend from the femoral tunnel 908 through the tibial tunnel 906, as shown in FIG. 14, and must be attached to the interior of the tibial tunnel 906 until biologic incorporation, i.e., osteo-integration of the graft ligament 801 within the tibial tunnel 906, is complete.

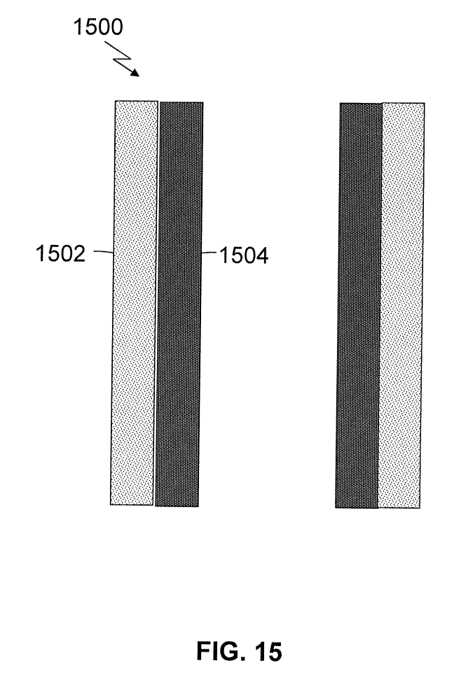

In one embodiment, the graft ligament 801 is secured in the tibial tunnel 906 using a bone-ligament-bone composite graft. As shown in FIG. 15, two corticocancellous bone grafts 1500 are obtained from either the patient or a donor. The corticocancellous bone grafts 1500 include softer cancellous bone 1502 on one side of the grafts 1500 and harder cortical bone 1504 on the other side of the grafts 1500. In another embodiment, the bone grafts 1500 may be made from artificial material, such as hydroxyapatite, ceramics, coral, or other suitable materials.

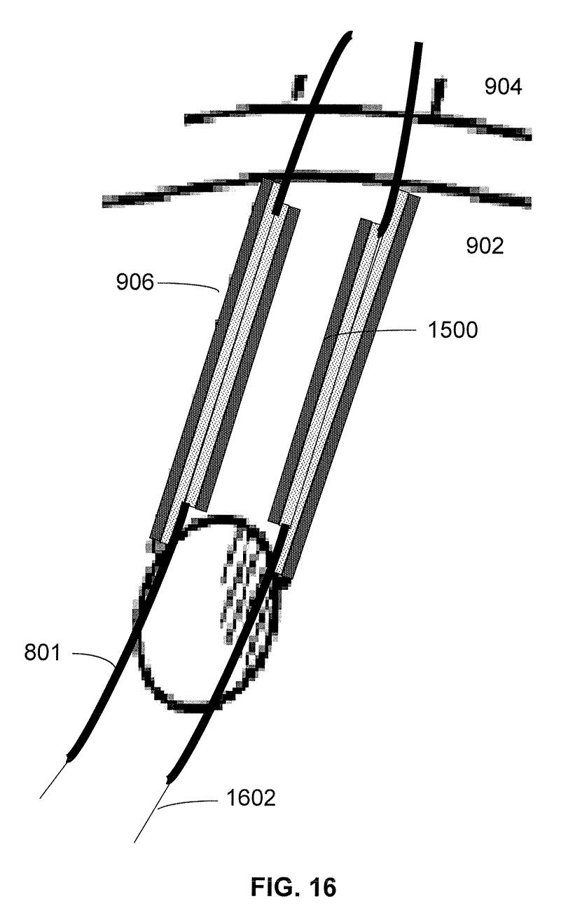

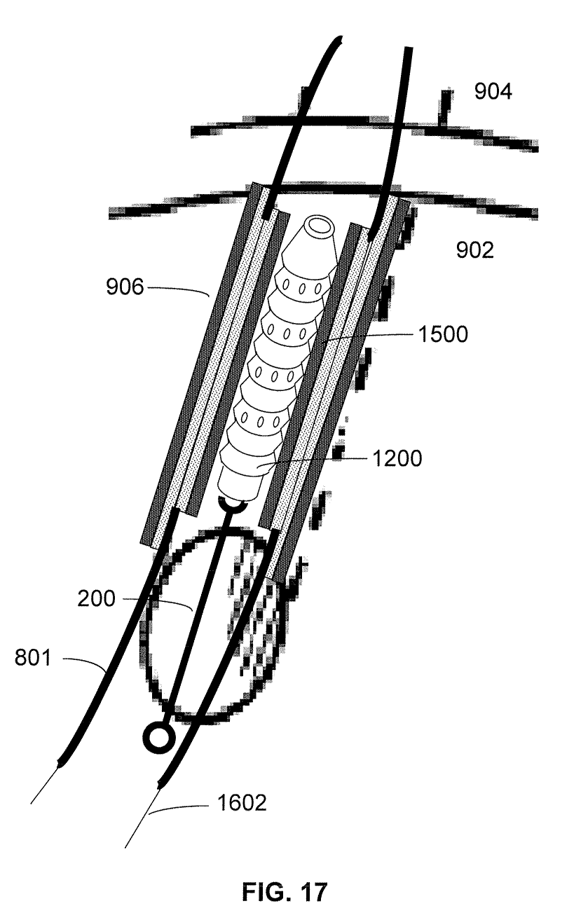

As shown in FIG. 16, the bone grafts 1500 are placed into the tibial tunnel 906, while the ends of the graft ligament 801 are held taut by stay sutures 1602. The graft ligament 801 is sandwiched between the cancellous bone 1502 of the bone grafts 1500 and the cancellous bone 910 in the tibial tunnel. The interference screw 1200 is then screwed into the tibial tunnel 906 between the bone grafts 1500 using the screwdriver 200 to wedge the bone grafts 1500 and the graft ligament 801 tightly into the tibial tunnel 906, as shown in FIG. 17. During the application of the interference screw 1200, the knee joint 900 may be held in full extension or in varying degrees of flexion.

In another embodiment, the bone grafts 1500 may be omitted and the interference screw 1200 may be driven into the tibial tunnel 906 and between the graft ligaments 801 to force the graft ligaments 801 against the walls of the tibial tunnel 906.

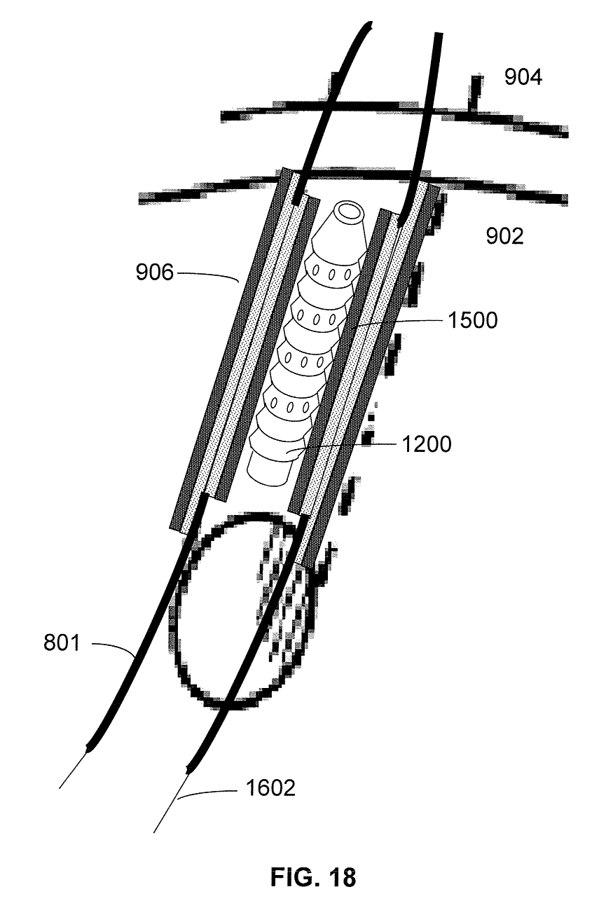

FIG. 18 shows the bone grafts 1500 and the interference screw 1200 seated in the tibial tunnel 906. The bone grafts 1500 serve to protect the graft ligaments 801 from abrasion by the threads 1230 of the interference screw 1200. The bone grafts 1500 should be at least as long as the threads 1230 of the interference screw 1200. However, in various embodiments, the bone grafts 1500 and interference screw 1200 may be of varying lengths and diameters according to the preference of the practitioner of the ligament reconstruction.

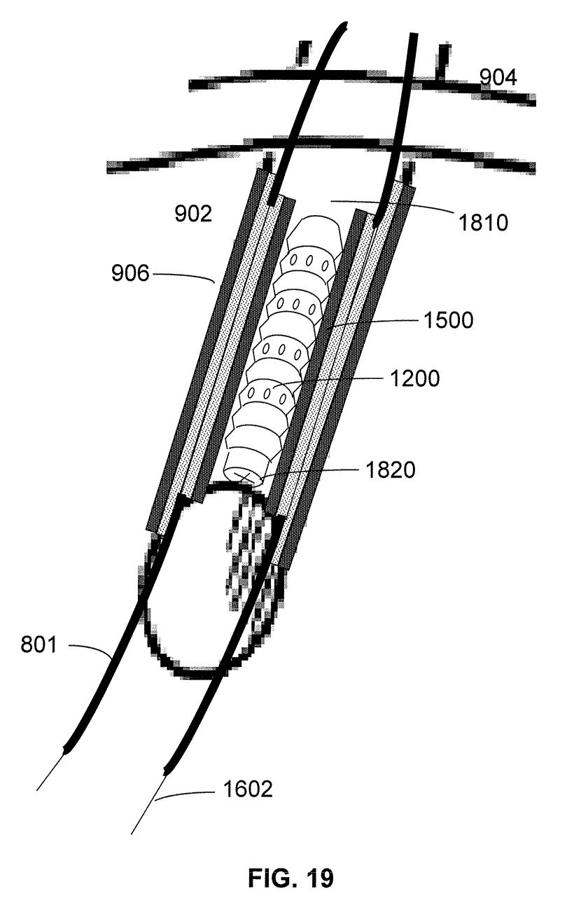

Finally, as shown in FIG. 19, an injectable or particulate graft 1810 is introduced into the tibial tunnel 906 between the bone grafts 1500 to effect osteo-integration. If the interference screw 1200 includes a cannula 1210 and perforations 1240, the injectable or particulate graft 1810 may be injected into the interference screw 1200 to be forced through the perforations 1240 and into contact with the bone grafts 1500. The cannula 1210 of the interference screw 1200 may then be capped with a screw plug 1820 screwed into the threads 1250 arranged on the inner surface 1262 of the wall 1260.

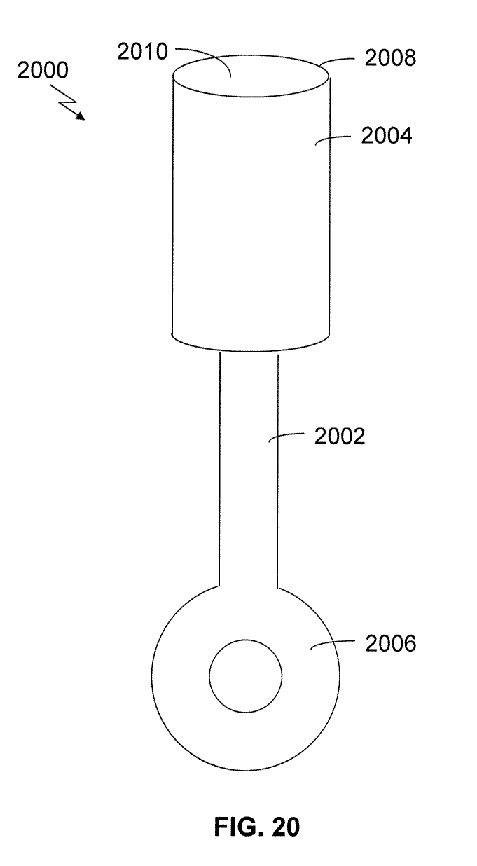

FIG. 20 is a diagram of a chisel 2000 that may be used to remove a screw from a bone tunnel. The chisel 2000 includes a handle 2006, a shaft 2002, and a cylindrical blade 2004 having a cannula 2010 and an edge 2008. The edge 2008 may be sharpened and may optionally be serrated. In one embodiment, the cannula 2010 may be deep enough to hold a screw to be removed from a bone tunnel. In another embodiment, the cannula 2010 may have a diameter that is slightly larger than the outer diameter of a screw to be removed from a bone tunnel. In use, the chisel 2000 may be inserted into a bone tunnel to envelop an implanted screw by tapping or rotation. The edge 2008 of the blade 2004 is used to break through bone and soft tissue that may have integrated with the screw so that the screw may be removed.

While embodiments of the invention have been described in detail in connection with embodiments known at the time, it should be readily understood that they are not limited to the disclosed embodiments. Rather, they can be modified to incorporate any number of variations, alterations, substitutions or equivalent arrangements not heretofore described. For example, while embodiments are described in connection with attaching a graft ligament to a femur and tibia in a procedure to reconstruct a damaged ACL, it should be understood that the embodiments described herein are not so limited and may be applied wherever surgical interference fixation is needed to fix soft tissue in a bone tunnel, for example, a PCL. Furthermore, while the various embodiments of methods are described as using a cannulated screw, crossbar, and/or interference screw of a particular embodiment, it should be understood than any of the cannulated screw, crossbar, and/or interference screw embodiments described herein may be used with the various methods.

What is claimed as new and desired to be protected by Letters Patent of the United States is:

1. A screw comprising:
a cylindrical wall having a leading end and a trailing end;
threads arranged on an outside surface of the cylindrical wall; and
a plurality of portals arranged in the cylindrical wall and adapted to receive one or more of a plurality of prongs of a cooperating screwdriver.

2. The screw of claim 1, wherein the screw comprises metal.

3. The screw of claim 1, wherein the screw comprises a biodegradable polymer.

4. The screw of claim 1, wherein the screw comprises a combination of metal and biodegradable polymer.

5. The screw of claim 1, wherein the plurality of portals comprises more than two portals.

6. The screw of claim 1, further comprising a plurality of perforations arranged in the cylindrical wall.

7. The screw of claim 1, wherein at least a portion of the outer surface of the cylindrical wall at the trailing end of the screw is smooth.

8. The screw of claim 1, further comprising a cannula extending from the leading end and through the trailing end of the screw.

9. The screw of claim 1, further comprising a cannula partially extending from the leading end and through the trailing end of the screw.

10. The screw of claim 8, further comprising a crossbar arranged at the leading end of the screw.

11. The screw of claim 10, wherein the crossbar comprises a handle having a first end portion and a second end portion, and said end portions of said handle are wider than the handle.

12. The screw of claim 11, wherein the end portions are spherical.

13. The screw of claim 12, wherein the end portions are not centered on the handle.

14. The screw of claim 12, wherein the crossbar comprises a bone graft.

15. The screw of claim 8, further comprising threads arranged on an inside surface of the cylindrical wall at a portion of the trailing end of the screw.

16. A screwdriver adapted to cooperate with the screw of claim 1, the screwdriver comprising a handle, a shaft, and a plurality of prongs adapted to fit one or more of the plurality of portals of the screw of claim 1.

17. A hood adapted to cover at least a portion of the prongs of the screwdriver of claim 16, the hood comprising an opening and comprising a material that is perforated when the plurality prongs of the screwdriver of claim 16 are inserted into the plurality of portals of the screw of claim 1.

18. A chisel adapted to remove the screw of claim 1, the chisel comprising a cylindrical blade and a cannula arranged in the cylindrical blade.

19. A method of attaching a graft ligament to a bone comprising:
forming a tunnel in the bone;
providing a screw comprising,
a cylindrical wall having an outer surface and an inner surface,
a cannula extending from a leading end and through the trailing end of the screw,
threads arranged on an outside surface of the cylindrical wall,
a plurality of portals arranged in the cylindrical wall and adapted to receive one or more of a plurality of prongs of a screwdriver, and
a crossbar arranged at the leading end of the screw;
arranging a graft ligament through the through hole of the screw and around the crossbar; and
screwing the screw and the graft ligament into the tunnel in the bone.

20. The method of claim 19, wherein the tunnel comprises only one opening and a distal end, and wherein the screw is screwed into the tunnel to cause the graft ligament to contact the distal end of the tunnel.

21. The method of claim 19, wherein forming the tunnel comprises forming a first tunnel through a tibia to a femur connected to a knee joint and forming a second tunnel into the femur and wherein the screw and the graft ligament are inserted through the first tunnel and into the second tunnel.

22. The method of claim 19, further comprising driving the screw and the graft ligament into the tunnel in the bone using a cooperating screwdriver, the screwdriver comprising a handle, a shaft, and a plurality of prongs adapted to fit into one or more of the plurality of portals of the screw.

23. The method of claim 22, further comprising:
covering the screwdriver with a hood;
inserting the screwdriver and hood into the tunnel in the bone;
inserting the plurality of prongs of the screwdriver into one or more of the plurality of portals of the screw; and
perforating the hood with the plurality of prongs.

24. A method of attaching a graft ligament to a bone comprising:
forming a tunnel in the bone;
providing a screw comprising,
a cylindrical wall having an outer surface and an inner surface,
threads arranged on an outside surface of the cylindrical wall, and
a plurality of portals arranged in the cylindrical wall and adapted to receive one or more of a plurality of prongs of a screwdriver;
providing a graft ligament in the tunnel; and driving
the screw into the tunnel using a cooperating screwdriver to fix the graft ligament into the tunnel.

25. The method of claim 24, further comprising arranging a bone graft in the tunnel so that the graft ligament is fixed between the bone graft and a wall of the tunnel.

26. The method of claim 24, wherein the screw further comprises a cannula extending from the leading end through the trailing end of the screw and a plurality of perforations arranged in the cylindrical wall, and wherein the method further comprises applying an injectable or particulate graft into the cannula of the screw and sealing the cannula of the screw.

27. The method of claim 24, further comprising driving the screw into the tunnel using a cooperating screwdriver, the screwdriver comprising a handle, a shaft, and a plurality of prongs adapted to fit into one or more of the plurality of portals of the screw.

28. The method of claim 25, further comprising:
covering the screwdriver with a hood;
inserting the screwdriver and hood into the tunnel;
inserting the plurality of prongs of the screwdriver into one or more of the plurality of portals of the screw; and
perforating the hood with the plurality of prongs.
